(19) United States
(12) Patent Application Publication
(10) Pub. No.: US 2026/0256489 A1
(43) Pub. Date: Sep. 3, 2026

(54) Surgical Cutting Accessory With Malleable Tube Assembly

(71) Applicant: Stryker European Operations Limited, Carrigtwohill, Co Cork (IE)

(72) Inventors: Conor O'Shea, Innishannon (IE); Jonathan Browne, Dublin (IE); Joshua Walsh, Midleton (IE); Daniel O'Brien, Charleville (IE); Jack O'Donovan, Clonakilty (IE); Nuala Nicolasi, Kilworth (IE); Branko Milankovic, Carrigtwohill (IE)

(73) Assignee: Stryker European Operations Limited, Carrigtwohill, Co Cork (IE)

(21) Appl. No.: 19/491,251

(22) PCT Filed: Jun. 14, 2024
(86) PCT No.: PCT/IB2024/055851

§ 371 Date: Dec. 8, 2025

Related U.S. Application Data
(60) Provisional application No. 63/521,423, filed on Jun. 16, 2023.

(51) Int. Cl. A61B 17/32; A61B 17/00

(52) U.S. Cl. CPC A61B 17/32002; A61B 2017/00946; A61B 2017/320024; A61B 2017/320032

(57) ABSTRACT

A cutting accessory for a surgical instrument. The cutting accessory includes a tube assembly (38) including an outer tube having at least one slotted region (50) configured to be bent to a shaped configuration and maintain the tube assembly in the shaped configuration. The slotted regions may be a proximal slotted region spaced apart from a distal slotted region to define a rigid intermediate segment, and a rigid distal segment of the tube assembly. The proximal or distal slotted regions may be at different rotational orientations. The spacing between adjacent slots of the proximal slotted region may be different than spacing between adjacent slots of the distal slotted region. The tube assembly may be shaped with an initial prebend at a predetermined angle and configured to be bent relative to the predetermined angle. The tube assembly may be a two-tube or three-tube arrangement.

### PRIORITY CLAIM

This application claims priority to and all the benefits of U.S. Provisional Patent Application No. 63/521,423, filed on Jun. 16, 2023, the entire contents being hereby incorporated by reference.

### BACKGROUND

Powered surgical cutting instruments are ubiquitous in the modern surgical suite and used to resect nearly all tissue types in nearly all anatomical locations. The form of the cutting instrument may be based in part on accessibility of the tissue to be resected. For orthopedic procedures, for example, the target tissue may be approached in a relatively straightforward manner, and the cutting instrument may include a straight shaft for on-axis resection. For more delicate procedures involving difficult-to-access anatomy, such as the ear, nose, and throat (ENT), the shaft may include at least one bend. Selection of an angle or curvature of the bend(s) is typically from a catalogue of cutting accessories, and therefore the predetermined bend angle(s) may not be the most suitable based on the patient-specific anatomy or surgical needs. Furthermore, the shafts may be rigid and therefore do not permit the surgeon to make on-the-fly adjustments during the surgical procedure.

United States Patent Publication No. 2013/0053830 to Edwards et al., published Feb. 28, 2013, discloses a tubular malleable segment on a shaft of a surgical instrument. Opposing ends of the tubular malleable segment are coupled to an outer tube within which an inner tube rotates. The tubular malleable segment provides for only limited malleable adjustment of a bend. Further, the tubular malleable segment relies on ductile materials, which may lose circularity with bending, increasing the likelihood of kinks or other damage to the shaft and potential premature failure of the surgical instrument. Still further, the tubular malleable segment may apply a bending moment on the inner tube due to the compliant nature of the ductile materials.

### SUMMARY

The present disclosure is directed to a cutting accessory for a surgical instrument that provides malleable adjustment to a tube assembly. The tube assembly may include one or more malleable regions. The malleable region(s) may be formed by a series of slots defining at least one malleable spine therebetween. The slots may be formed with several characteristics to impart a desired flexibility and permit a user to bend the tube assembly to a shaped configuration with the sturdiness to maintain the tube assembly in the shaped configuration. The slot characteristics may include kerf, segment length, uncut angle, cut angle, upper slot angle, and lower slot angle. among others. The slot characteristics or cut pattern may be specifically designed to maintain circularity of the outer tube, providing for greater bend angles and improved component lifespan. The maximum bend angle achievable with the cutting accessory is greater than known devices, and similarly sharper curvatures are achievable.

Advantages of the cutting accessory are several. First, a bend angle imparted to the tube assembly may be varied and specifically tailored to the anatomy to be accessed. In other words, multiple procedural approaches may be provided with a single device, reducing equipment costs as well as environmental waste. The bend(s) imparted to the tube assembly are particularly well-suited for endoscopic procedures involving difficult-to-access anatomy, for example, ENT procedures with trans-nasal or trans-oral approaches. One, two, three, four or more bends may be effectuated, and proximal and distal malleable regions spaced apart from one another may facilitate the bends being approximated at the desired axial location(s) along the tube assembly. The bend may be removed for subsequent steps of the surgical procedure without affecting the performance of the surgical instrument.

In certain implementations, the cutting accessory includes an outer hub, and a drive hub is rotatably disposed within the outer hub. A tube assembly extends distally from the outer hub and includes an outer tube, an inner tube, and, optionally, an intermediate tube. The outer tube is coupled to the outer hub, the intermediate tube is coaxially disposed within the outer tube, and the inner tube is coaxially disposed within the intermediate tube and coupled to the drive hub. A cutting tip of the tube assembly may be a microdebrider or a bur, or other powered or manual cutting instrument such as a curette, rasp, blade tip, trephine, brush, a screwdriver, endoscopic camera, light assembly, or the like.

The outer tube includes at least one slotted region in which the slots form at least one malleable spine. The inner tube includes at least one flexible region corresponding at least to an axial location of slotted region(s). In implementations in which there is an intermediate tube, the intermediate tube also includes at least one least one flexible region corresponding at least to the axial location of the slotted region(s) and the axial location of the flexible region(s) of the inner tube. The slotted region may include an upper series of slots and a lower series of slots spaced apart from the upper series of slots by the malleable spine. Characteristics of the bending of the tube assembly may also be based on the segment length, and the segment lengths may be the same or varied along the length of the slotted region. The uncut and cut angles may be selectively designed to afford a desired combination of flexibility and permit the user to bend the tube assembly to the shaped configuration with the sturdiness to maintain the shaped configuration once bent. It is contemplated that the cut angle of the upper series of slots may be different than the cut angle of the lower series of slots, or vice versa. The uncut angle associated with one of the malleable spines may be different than the other one of the malleable spines, in which case the slots may be radially offset. Lastly, varying the upper slot angle and/or the lower slot angle provides for more complex geometries to achieve a desired combination of flexibility and sturdiness.

The slotted region may extend along 25, 50, 75, 90, or more percent of the length of the tube assembly distal to the outer hub. Alternatively, there may be more than one slotted region. A proximal slotted region may be spaced apart from a distal slotted region. One or both of the proximal slotted region and the distal slotted region may include any of the characteristics of the slots described throughout the present disclosure. The slot characteristics of the proximal slotted region may be the same or different than those of the distal slotted region. A length of the proximal slotted region may be the same or different than a length of the distal slotted region.

In certain implementations, it may be desirable to have more two or more bends in which the cutting tip is offset in at least two directions relative to the tube assembly in the straight configuration. The proximal slotted region may be oriented at a proximal radial angle, and the distal slotted region may be oriented at a distal radial angle different than the proximal radial angle. In certain implementations, the spacing between the slots differs along the length of the outer tube. For example, the spacing between adjacent slots of the series of slots of the proximal slotted region is greater than spacing between adjacent slots of the series of slots of the distal slotted region. In certain implementations, the tube assembly may be formed with an initial prebend. In certain implementations, the tube assembly may include an initial fixed prebend with slotted region(s) positioned distal to the initial fixed prebend.

In certain implementations, the intermediate tube facilitates rotational adjustment of the cutting window. The cutting accessory may include an actuator coupled to the outer hub and operably coupled to the intermediate tube. The coupling may be facilitated by suitable gearing within the outer hub, for example, bevel gearing, worm gearing, or the like. An input to the actuator is configured to rotate the intermediate tube relative to the outer tube, and the inner tube. Rotation of the intermediate tube rotates the cutting window about the longitudinal axis.

In certain implementations, the cutting accessory includes a sensor coupled to the tube assembly and disposed distal to the malleable region. The sensor may be an electromagnetic (EM) sensor or other suitable tracking technology that does not require line-of-sight. A lead couples the sensor to electronic subcomponents within the outer hub. The lead may extend proximally from the sensor. The lead may be a twisted wire pair to reduce interference from the sensor. The sensor and the lead may be secured to the outer tube with a sheath.

Therefore, according to a first aspect of the present disclosure, the cutting accessory includes the outer hub configured to be coupled with the handpiece. The drive hub is rotatably disposed within the outer hub and configured to be operably coupled to the motor. The tube assembly includes an outer tube coupled to and extending distally from the outer hub, an intermediate tube coaxially disposed within the outer tube and extending distally from the outer hub, an inner tube coupled to the drive hub and coaxially disposed within the intermediate tube, and a cutting tip disposed on the inner tube. Each of the intermediate tube and the inner tube includes at least one flexible region. The outer tube is formed with at least one slotted region in which a series of slots form a malleable spine configured to be bent and/or rebent by a user to a shaped configuration and maintain the tube assembly in the shaped configuration.

According to a second aspect of the present disclosure, the cutting accessory includes the outer hub configured to be coupled with the handpiece. The drive hub is rotatably disposed within the outer hub and configured to be operably coupled to the motor. The tube assembly includes an outer tube coupled to and extending distally from the outer hub, an inner tube coupled to the drive hub and coaxially disposed within the outer tube, and a cutting tip disposed on the inner tube, wherein the inner tube comprises at least one flexible region. The outer tube is formed with a proximal malleable region that is spaced apart from a distal malleable region to define a rigid proximal segment, a rigid intermediate segment, and a rigid distal segment of the tube assembly. The proximal and distal malleable regions are configured to be independently bent and/or rebent by a user to a shaped configuration and maintain the tube assembly in the shaped configuration.

According to a third aspect of the present disclosure, the cutting accessory includes the outer hub configured to be coupled with the handpiece. The drive hub is rotatably disposed within the outer hub and configured to be operably coupled to the motor. The tube assembly includes an outer tube coupled to and extending distally from the outer hub, an inner tube coupled to the drive hub and coaxially disposed within the outer tube, and a cutting tip disposed on the inner tube. The inner tube includes at least one flexible region. The outer tube is formed with a proximal slotted region and a distal slotted region in which a series of slots respectively form a proximal malleable spine and a distal malleable spine configured to be independently bent and/or rebent by a user to a shaped configuration and maintain the tube assembly in the shaped configuration. Spacing between adjacent slots of a series of slots of the proximal slotted region is greater than spacing between adjacent slots of a series of slots of the distal slotted region.

According to the fourth aspect of the present disclosure, the cutting accessory includes the outer hub configured to be coupled with the handpiece. The drive hub is rotatably disposed within the outer hub and configured to be operably coupled to the motor. The tube assembly includes an outer tube coupled to and extending distally from the outer hub, and an inner tube coupled to the drive hub and coaxially disposed within the outer tube. The inner tube includes at least one flexible region, and wherein the outer tube is formed with a malleable region. The malleable region is shaped with an initial prebend at a predetermined angle and configured to be bent and/or rebent by a user to a shaped configuration about the predetermined angle and maintain the tube assembly in the shaped configuration.

According to a fifth aspect of the present disclosure, the cutting accessory includes the outer hub configured to be coupled with the handpiece. The drive hub is rotatably disposed within the outer hub and configured to be operably coupled to the motor. The tube assembly includes an outer tube coupled to and extending distally from the outer hub, and an inner tube coupled to the drive hub and coaxially disposed within the outer tube, wherein the inner tube comprises at least one flexible region. The outer tube is formed with an initial fixed prebend, and a malleable region positioned distal to the initial fixed prebend. The outer tube is further formed with at least one slotted region in which a series of slots form a malleable spine configured to be bent and/or rebent by a user to a shaped configuration and maintain the tube assembly in the shaped configuration.

### BRIEF DESCRIPTION OF THE DRAWINGS

FIG. 1 is a perspective view of a powered surgical cutting instrument. A cutting accessory is removably coupled to a handpiece. The cutting accessory includes a tube assembly having at least one slotted region, and a malleable spine.

FIG. 2 is a perspective view of an implementation of the tube assembly.

FIG. 3 an elevation view of a distal portion of the malleable tube assembly of FIG. 2.

FIG. 4 is a perspective view of an implementation of the tube assembly in which a distal slotted region is spaced apart from a proximal slotted region by a rigid intermediate segment.

FIG. 5 is a perspective view of an implementation of the tube assembly in which the distal slotted region is oriented radially offset relative to the proximal slotted region.

FIG. 6 is a perspective view of an implementation of malleable tube assembly in which spacing between adjacent slots is different between slotted regions.

FIG. 7 is an elevation view of an implementation of the tube assembly in which the malleable region is shaped with at least one initial prebend.

FIG. 8 is an elevation view of an implementation of the tube assembly in which the malleable region is distal to at least one rigid prebend.

FIG. 9 is an elevation view of an implementation of the tube assembly in which the malleable region is distal to at least one rigid prebend. The malleable region is of sufficient length to provide for a compound bend distal to the rigid prebend.

FIG. 10 is an implementation of the tube assembly in which a sensor is positioned distal to the malleable region. A sheath is disposed over the sensor and a wire lead that is coupled to the sensor.

FIG. 11 is a detailed view of the implementation of the tube assembly of FIG. 10 within circle 11-11.

FIG. 12 is an elevation view of a bending apparatus configured to bend the tube assembly of the cutting accessory.

### DETAILED DESCRIPTION

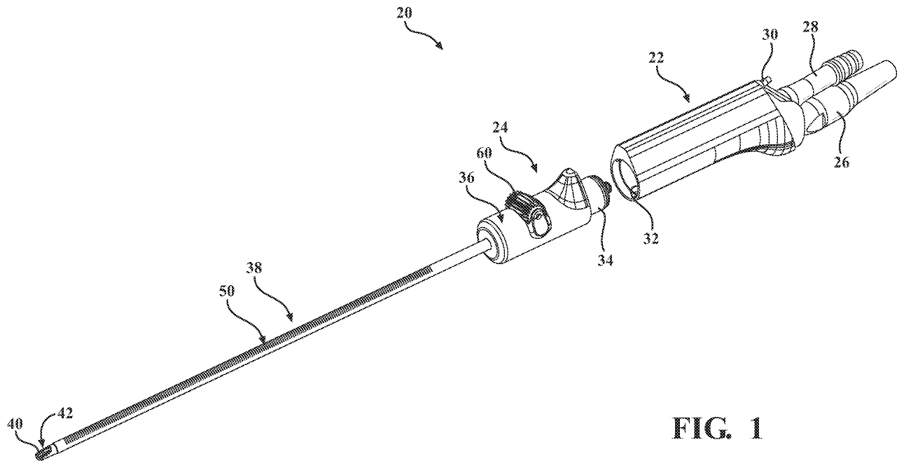

FIG. 1 shows a surgical cutting instrument 20 including a handpiece 22 and a cutting accessory 24. The handpiece 22 is a capital component; i.e., a component configured to be sterilized and reused over many surgical procedures. The cutting accessory 24 may be disposed of after a single use, or it may be manufactured to be sterilizable and reusable as well. The handpiece 22 includes a power port or power cord 26, a suction port 28, and, optionally, an irrigation port 30. A motor (not shown) within the handpiece 22 is driven by power transmitted through the power cord 26, which is configured to be removably coupled to a power source. The power source may be a surgical console, for example, a powered instrument driver sold under the tradename CORE by Stryker Corporation (Kalamazoo, Mich.). The suction port 28 is configured to removably receive a suction tube to establish a suction path between a cutting window 40 of a cutting tip 42 and a vacuum source (not shown) with which the suction tube is configured to be removably coupled. One suitable vacuum source is disposed on a waste management system sold under the tradename Neptune by Stryker Corporation. In certain implementations, the irrigation port 30 is configured to removably receive an irrigation tube to establish an irrigation path between the cutting tip 42 and a source of irrigation liquid (not shown). An irrigation pump is configured to direct the irrigation liquid through the handpiece 22 and through the cutting accessory 24 to be discharged from the cutting tip 42 at the surgical site. Alternatively, the vacuum source and/or the irrigation pump may be integrated on the surgical console. The suction and irrigation paths of the surgical cutting instrument 20 may be at least similar to those disclosed in commonly owned International Publication No. WO 2021/224862, published Nov. 11, 2021, and commonly owned International Publication No. WO 2022/123535, published Jun. 16, 2022, the entire contents of each being hereby incorporated by reference.

The handpiece 22 defines at least one opening or cavity 32 configured to removably receive at least a portion of the cutting accessory 24. The cutting accessory 24 may include a drive hub 34 configured to be directed to within the cavity 32 and include at least one interfacing geometry configured to be operably coupled with the motor. The handpiece 22 and/or the cutting accessory 24 include complementary coupling features (not identified) to releasably secure the cutting accessory 24 to the handpiece 22. The coupling features may be a latch or other suitable interlocking geometries, for example, those disclosed in the aforementioned International Publication No. WO2021/224862.

The cutting accessory 24 includes a housing, also referred to herein as an outer hub 36. The outer hub 36 may be contoured for ergonomic grasping and manipulation. The drive hub 34 is rotatably disposed within the outer hub 36. A tube assembly 38 extends distally from the outer hub 36 and includes an outer tube 44 and inner tube 46, and, optionally, an intermediate tube 48. The outer tube 44 is coupled to the outer hub 36, the intermediate tube 48 is coaxially disposed within the outer tube 44, and the inner tube 46 is coaxially disposed within the intermediate tube 48 and coupled to the drive hub 34. Each of the tubes 44, 46, 48 may extend distally from the outer hub 36. In other words, the tubes 44, 46, 48 may include proximal ends that are disposed within an interior of the outer hub 36. Stated differently, the tubes 44, 46, 48, and in particular the outer tube 44, may not merely be a tubular segment that is external to the outer hub 36.

The tube assembly 38 includes the cutting tip 42. In certain implementations, the cutting tip 42 is a toothed tip coupled to, or disposed on, the inner tube 46. The intermediate tube 48 may define the cutting window 40 with the inner tube 46 rotatable within the intermediate tube 48. As a result, with the drive hub 34 operably coupled to the motor, the motor rotates the inner tube 46 to cause a cutting edge to shear or debulk the tissue within the cutting window 40. The resected tissue is suctioned through the cutting window 40 and into the suction path. In another exemplary implementation, the cutting tip 42 is a bur head coupled to the inner tube 46. The intermediate tube 48 may define a tubular distal end from which the bur head extends, and, optionally, a hood may at least partially surround the bur head. With the drive hub 34 operably coupled to the motor, the motor rotates the inner tube 46 to cause the bur head to resect the tissue. It is contemplated that the cutting tip 42 may assume other suitable forms, and the surgical cutting instrument need not be electrically powered. For example, the implementations of the tube assembly 38 to be described may be used with manual cutting instruments such as a curette, rasp, blade tip, trephine, brush, or the like, or non-cutting manual, or powered instruments such as a screwdriver, endoscopic camera, light assembly, or the like.

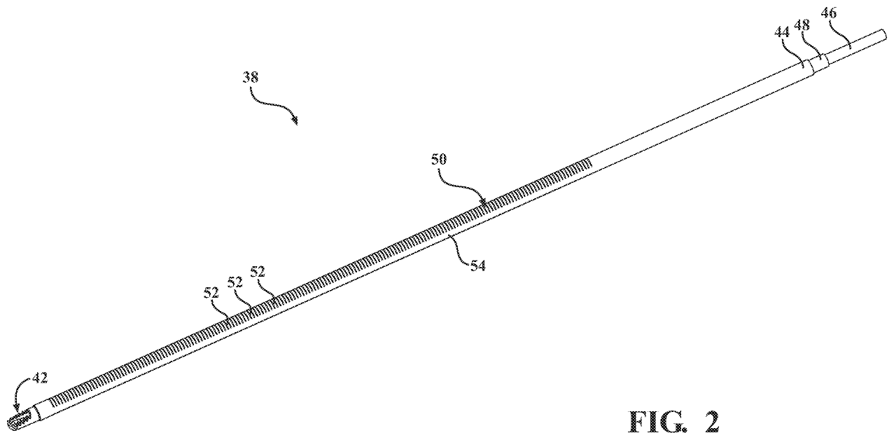

The tube assembly 38 is configured to be bent and/or rebent by a user to a shaped configuration and be maintained in the shaped configuration. More particularly, at least a portion of the tube assembly 38 includes a malleable region to permit the user to bend it to the shaped configuration, after which the tube assembly 38 is sufficiently sturdy to maintain the shaped configuration despite axial and radial forces associated with deploying the cutting tip 42 at the surgical site. Referring to FIG. 2, the outer tube 44 includes at least one slotted region 50 in which a series of slots 52 form at least one malleable spine 54, and the inner tube 46 includes at least one flexible region (not identified) corresponding at least to an axial location of the slotted region(s) 50. In implementations in which there is an intermediate tube 48, the intermediate tube 48 also includes at least one flexible region (not identified) corresponding at least to the axial location of the slotted region(s) 50, and the axial location of the flexible region(s) of the inner tube 46. The malleable spine 54 is configured to be bent and/or rebent by a user to a shaped configuration, and be maintained in the shaped configuration, and the flexible regions of the inner and intermediate tubes 46, 48 are configured to conform to the shaped configuration maintained by the outer tube 44. The flexible regions may be accomplished through several suitable manners, such as those disclosed in the aforementioned International Publication No. WO2022/123535. In one example, the flexible region includes castellated segments interlocked with one another to define slots. The interlocking of the segments is configured to transmit torque with rotation of the inner tube 46 by the motor. Additionally, or alternatively, the inner tube 46 may include helical, spiral, wound, or braided characteristics configured to transmit torque about the bend(s) of the tube assembly 38 in the shaped configuration. The flexible regions may be formed by cut geometries that are different than the series of slots 52 of the slotted region 50 of the outer tube 44.

As used herein, the series of slots 52 indicates at least two slots axially spaced apart from one another to provide for bending, but the exemplary implementations typically include many slots being disposed along the outer tube 44 in numerous configurations to be described. Additionally, alternative configurations include one or more helical slots extending about the outer tube 44 with a suitable pitch, and an orthogonal cut pattern of alternating axial and circumferential slots, the latter being shown empirically to reduce bending stiffness to approximately 25 percent of that of an uncut tube. The slots 52 need not be linear but may instead be formed in curved or zig-zagged arrangements or other complex geometries.

Owing to the material forming the tube assembly 38, the malleable spines 54 are configured to plastically deform in a reversible manner, thus maintaining the tube assembly 38 in the shaped configuration. Suitable materials may include stainless steel, aluminum, or another biocompatible metal, plastic, polymer, or composite having an appropriate yield strength to provide malleability. Exemplary metals may include 316L stainless steel and 3003 aluminum having yield strengths of 515 megapascals (MPa) and 186 MPa, respectively. The slotted region 50 may be fabricated through any suitable manufacturing techniques, including but not limited to laser cutting, electrical discharge manufacturing (EDM), three-dimensional printing, and the like.

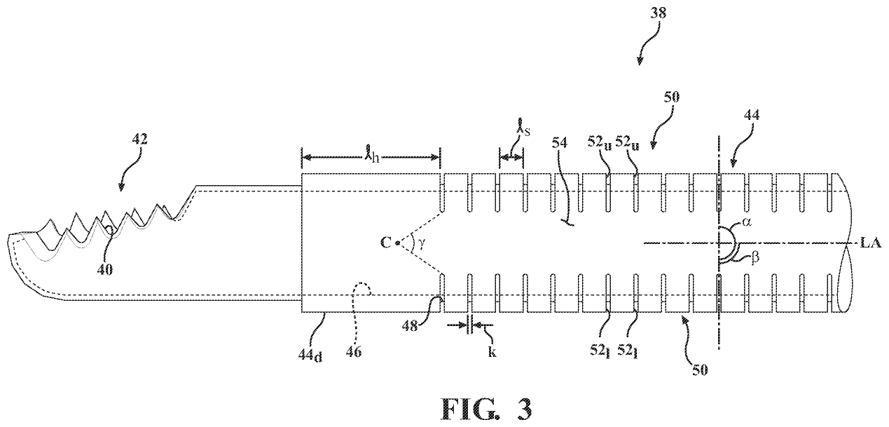

FIGS. 2 and 3 viewed in combination show the slotted region 50 including an upper series of slots 52u, and a lower series of slots 52l spaced apart from the upper series of slots 52u by the malleable spine 54. Another malleable spine (not visible) is disposed on the opposing side of the outer tube 44. Therefore, the cutting accessory 24 provides for the user to bend the tube assembly 38 upwards in which the upper series of slots 52u narrow and the lower series of slots 52l widen, and/or downwards in which the lower series of slots 52/narrow and the upper series of slots 52u widen. The extent by which the user may bend the tube assembly 38 is, in part, a function of a kerf (k) of the slots 52, with larger kerfs providing for greater adjustment of the bend angles. As used herein, the kerf is a width of the slot. The kerfs of the slots 52 may be within the range of approximately 0.02 to 0.08 millimeters, and more particularly, approximately 0.05 millimeters.

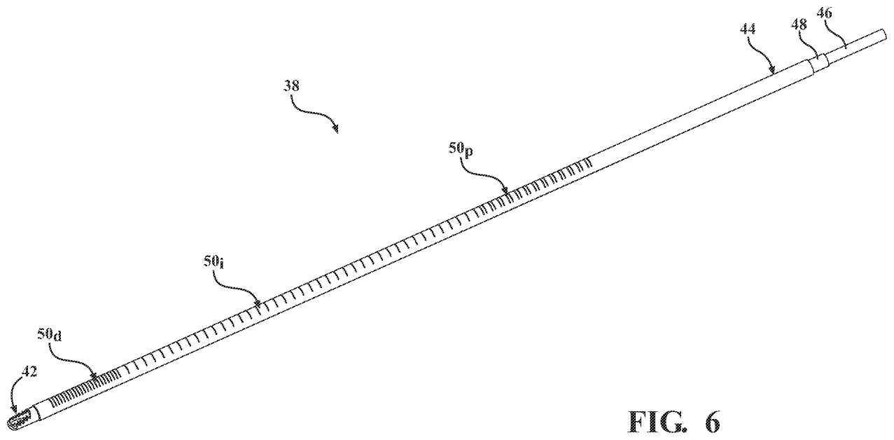

Referring to FIG. 3, further characteristics of the bending of the tube assembly 38 may be based on the segment length (lₛ), an uncut angle (γ) and corresponding cut angle (not shown?), an upper slot angle (α), and a lower slot angle (β). The segment length may be considered a length of the outer tube 44 between adjacent slots 52; i.e., the spacing between adjacent slots 52. The segment lengths may be the same along the length of the slotted region 50 (see FIGS. 2-5) or varied (see FIG. 6). For example, the segment lengths may be within the range of approximately 0.5 to 2.5 millimeters, and more particularly within the range of approximately 0.1 to 2.0 millimeters. Furthermore, the segment lengths of the upper series of slots 52u may be the same or different than the segment length of the lower series of slots 52l. Still further, the segment lengths may be the same along the length of the slotted region 50, but the axial positions of the upper series of slots 52u and the lower series of slots 52l may be “staggered” (e.g., not axially coplanar as shown in FIG. 3).

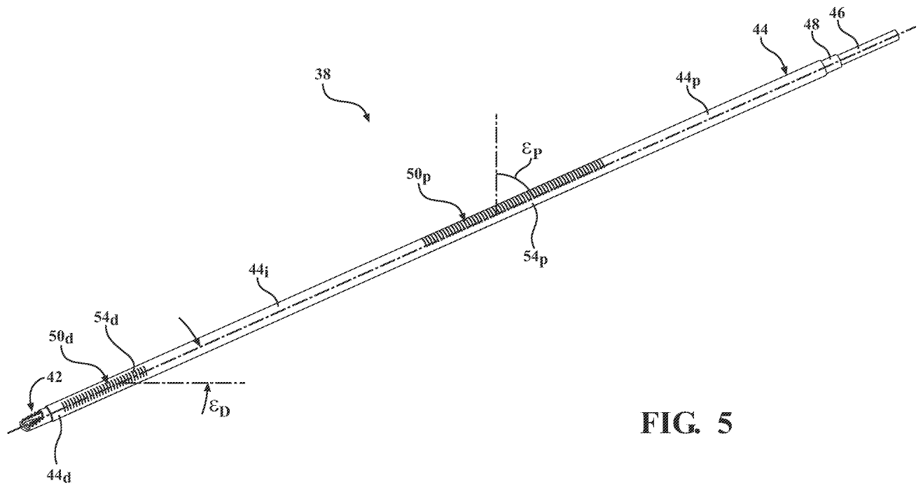

The slots 52 extend circumferentially about a portion of the outer tube 44. The uncut angles may be considered respective arcs by which the malleable spines 54 subtend the outer tube 44, and the cut angles may be considered respective arcs by which the upper series of slots 52u and the lower series of slots 52l subtends the outer tube 44. A summation of the uncut angles and the cut angles equals 360 degrees. For explanatory purposes, FIG. 3 represents a coaxial center point (C) of the tube assembly 38, and the uncut angle may be measured between an end of one of the upper slots 52u and an end of a corresponding one of the lower slots 52l. The cut angles may be measured between opposing ends of a singular one of the slots 52. In one example, the uncut angle may be approximately between 40 and 100 degrees, and more particularly within the range of approximately 60 to 80 degrees. The cut angle may be approximately between 60 and 120 degrees, and more accurately within the range of approximately 80 to 100 degrees. Other values are within the scope of the present disclosure. Generally, the uncut and cut angles may be selectively designed to afford a desired combination of flexibility and permit the user to bend the tube assembly 38 to the shaped configuration with the sturdiness to maintain the shaped configuration once bent. It is contemplated that the cut angle (α) of the upper series of slots 52u may be different than the cut angle (β) of the lower series of slots 52l, or vice versa, in which case certain slots 52 may be formed “deeper” into the outer tube 44 with larger cut angles. It is further contemplated that the uncut angle associated with one of the malleable spines 54 may be different than the uncut angle (γ) of another one of the malleable spines 54, in which case the slots 52 may be radially offset (see FIG. 5).

The upper slot angle and the lower slot angles may be defined between the longitudinal axis (LA), and a plane extending through a respective one of the upper slot 52u and the lower slot 52l. FIG. 3 shows the upper and lower slot angles being 90 degrees. In other words, the slots 52 of the illustrated implementation are transverse to the longitudinal axis of the tube assembly 38. In alternative variants, the upper slot angle and/or the lower slot angle may be less than 90 degrees such that the series of slots 52 is oriented proximally, or greater than 90 degrees such that the series of slots 52 is oriented distally. The upper slot angle need not be the same as the lower slot angle. Further, in variants in which the segment lengths are different between the upper series of slots 52u and the lower series of slots 52l, varying the upper slot angle and/or the lower slot angle provides for more complex geometries to achieve the desired combination of flexibility and sturdiness. Empirical data has realized the advantageous designs disclosed herein of achieving bend angles of greater than 60 degrees while withstanding the load of at least 15 Newtons on the cutting tip 42 in a shaped configuration.

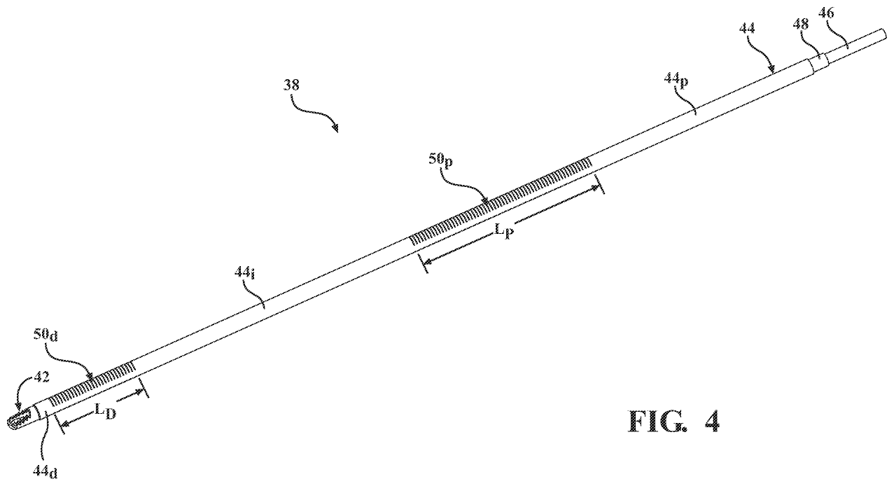

FIG. 2 in view of FIG. 1 shows the slotted region 50 extending along nearly an entirety of an exposed length of the outer tube 44 that is distal to the outer hub 36. In certain implementations, the length of the tube assembly 38 distal to the outer hub 36 may be between 20 to 50 centimeters depending on the clinical application. The slotted region 50 may extend along 25, 50, 75, 90, or more percent of the length of the tube assembly 38 distal to the outer hub 36. In another implementation shown in FIG. 4, the at least one slotted region 50 is a proximal slotted region 50p, and the outer tube 44 is further formed with a distal slotted region 50d that is spaced apart from the proximal slotted region 50p. The proximal slotted region 50p and the distal slotted region 50d define a rigid proximal segment 44p, a rigid intermediate segment 44i, and a rigid distal segment 44d of the tube assembly 38. The rigid distal segment 44d is also referred to herein as a tube head. Such an implementation is particularly well-suited for laryngeal and other clinical procedures in which the tube assembly 38 is relatively large and an intermediate portion of the tube assembly 38 is unlikely to require bending. A length of the tube head (lₙ) (see FIG. 3) may be relatively small, for example, one centimeter or less. One or both of the proximal slotted region 50p and the distal slotted region 50d may include any of the characteristics of the slots 52 described throughout the present disclosure, namely kerf, segment length, uncut angle, cut angle, upper slot angle, and lower slot angle. The slot characteristics of the proximal slotted region 50p may be the same or different than those of the distal slotted region 50d.

A length of the proximal slotted region 50p may be the same or different than a length of the distal slotted region 50d. The illustrated implementation of FIG. 4 shows the length of the proximal slotted region 50p (L_{P}) being greater than that of the length of the distal slotted region 50d (LD). Similarly, respective lengths of the rigid proximal segment 44p, the rigid intermediate segment 44i, and the rigid distal segment 44d of the tube assembly 38 may be designed to locate the proximal slotted region 50p and the distal slotted region 50d in desired axial locations along the outer tube 44. The axial location of the proximal slotted region 50p and the distal slotted region 50d in the illustrated implementation may be exemplary of those for a laryngeal application. It is appreciated that the cutting window 40 may be modified from what is shown in FIG. 4 based on the anticipated bend angles of the distal slotted region 50d.

As mentioned previously, the upper series of slots 52u and the lower series of slots 52l generally allow for the user to bend the tube assembly 38 upwardly and/or downwardly. In other words, the tube assembly 38—after being bent or rebent-typically remains coplanar along a vertical plane corresponding to the user holding the surgical cutting instrument 20 in the upright position. In certain implementations, it may be desirable to have two or more bends in which the cutting tip 42 is offset in at least two directions relative to the tube assembly 38 in the straight configuration. FIG. 5 shows an implementation of such an arrangement in which the series of slots 52 of the proximal slotted region 50p are formed to orient a proximal malleable spine 54p on a first rotational orientation. The series of slots 52 of the distal slotted region 50d are formed to orient a distal malleable spine 54d in a second rotational orientation different than the first rotational orientation. Relative to the vertical plane extending vertically through the longitudinal axis, the proximal slotted region 50p may be oriented at a proximal radial angle (ε_{D}), and the distal slotted region 50d may be oriented at a distal radial angle (ε_{D}) different than the proximal radial angle. In the illustrated implementation the proximal radial angle is zero degrees. In other words, the upper series of slots 52u and the lower series of slots 52l (not visible) previously described are aligned along the vertical plane. The distal radial angle may be non-zero. For example, the distal radial may be within the range of approximately 10 to 170 degrees. FIG. 5 shows the distal radial angle at approximately 45 degrees, or the 2 o'clock position—and it is understood that another series of slots (not visible) may be disposed on the opposing side of the outer tube 44. Such an arrangement permits the user to bend the rigid distal segment 44d relative to the rigid intermediate segment 44i in lateral directions.

Depending on the other characteristics of the slots 52 of the distal slotted region 50d, some upward and downward bendability may also be provided in addition to the lateral bendability. For convention, the proximal and distal radial angles may instead be measured between the vertical plane and the proximal and distal malleable spines 54p, 54d, respectively. The illustrated arrangement is a non-limiting example, and it should be appreciated that all other aspects of the present disclosure may be incorporable on the tube assembly 38 having one or more slotted regions 50 that are radially offset. In one example, the tube assembly 38 need not have the rigid intermediate segment 44i, but rather the slotted region 50 may extend for nearly an entirety of the exposed length of the tube assembly 38—and, at a suitable location, one of the slots 52 in the series of slots 52 is radially offset, or “clocked”, relative to the slot 52 immediately adjacent. In another example, the proximal slotted region 50p may be radially offset relative to the vertical plane with the distal slotted region 50d being aligned with the vertical plane, or radially offset at a distal radial angle different than the proximal radial angle. It is contemplated that the hub 36 and/or the tube assembly 38 may include visual indicia indicative of a direction of the bend. The visual indicia may be, for example, a laser etching, printed marking, or the like.

Referring now to FIG. 6, another implementation of the tube assembly 38 is shown in which spacing between the slots 52 differs along the length of the outer tube 44. In other words, the segment lengths are different (see FIG. 3). This aspect is combinable with any of the other aspects of the present disclosure. In an exemplary configuration, the spacing between adjacent slots of the series of slots 52 of the proximal slotted region 50p is greater than spacing between adjacent slots of the series of slots 52 of the distal slotted region 50d. For example, laryngeal procedures typically require a sharper curve on the distal bend and a more gradual curve on the proximal end. The illustrated implementation shows the spacing between pairs of slots 52 of the proximal slotted region 50p being greater than the uniform spacing of the distal slotted region 50d. As a result, the user may bend the distal slotted region 50d to have a greater radius of curvature than that of the proximal slotted region 50p. In an optional variant, FIG. 6 shows an intermediate slotted region 50i in which the spacing between adjacent slots of the series of slots 52 of the intermediate slotted region 50i is different than the spacing of each of the proximal and distal slotted regions 50p, 50d. As mentioned, the tube assembly 38 for laryngeal procedures may be relatively large with an intermediate portion unlikely to require more than minimal bending. The intermediate slotted region 50i reflects such considerations by permitting some bending but otherwise being sturdier than the proximal and distal slotted regions 50p, 50d. More particularly, the spacing of intermediate slotted region 50i is greater than the spacing of each of the proximal and distal slotted regions 50p, 50d, and thus the is generally constrained from bending as sharply as the proximal and distal slotted regions 50p, 50d.

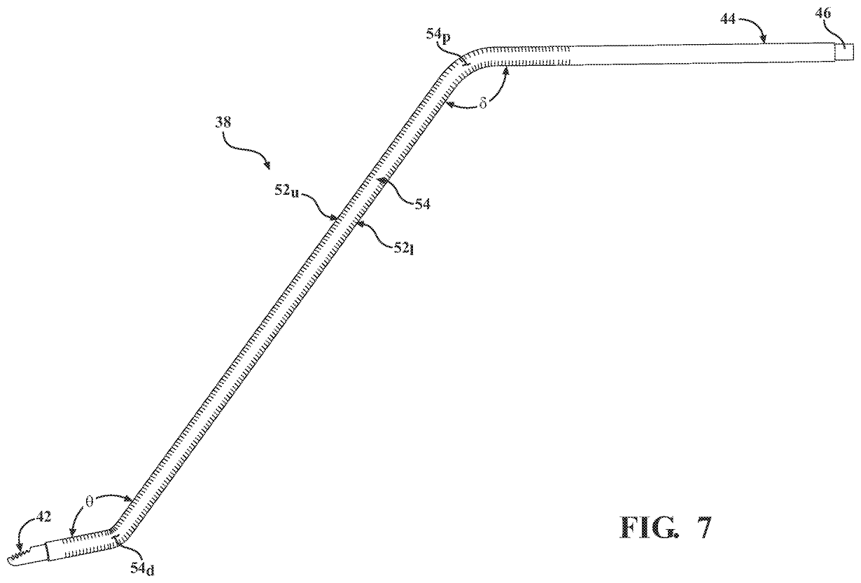

In certain implementations, the tube assembly 38 is formed in an initial straight configuration, after which the slotted region(s) 50 may be bent as desired. The range by which the slotted region 50 may be bent is relative to the longitudinal axis of the tube assembly 38 in the straight configuration. In other implementations, in which it is known that bending is necessary (e.g., laryngeal procedures), the tube assembly 38 may be formed with an initial prebend that approximates the likely-to-be-shaped configuration indicated for the procedure. Then, the slotted region's 50 range of bending is relative to the initial prebend, thereby affording the user a greater range of more relevant bend angles. Referring now to FIG. 7, an implementation of the tube assembly 38 is shown in which the proximal malleable spine 54p includes a proximal initial prebend at a proximal predetermined angle (δ), and the distal malleable spine 54d includes a distal initial prebend at a distal predetermined angle (θ). The proximal and distal predetermined angles may be the same or different, and of any suitable magnitude. For example, within the range of approximately 45 to 175 degrees, and more particularly within the range of approximately 90 to 135 degrees. As a result, the proximal and distal malleable spines 54p, 54d are configured to be bent and/or rebent to the shaped configuration about the proximal and distal predetermined angles, respectively.

It is understood that either the proximal malleable spine 54p or the distal malleable spine 54d may include the initial prebend, with the other being in the initial straight configuration. For example, the distal malleable spine 54d may include the initial prebend-rigid (i.e., an absence of the slotted region 50) or malleable-with the proximal malleable spine 54p being in the initial straight configuration. In still another variant, the proximal initial prebend may be rigid, and the distal slotted region 50d spaced apart from the rigid proximal prebend by a rigid intermediate segment. Implementations including the initial prebend may also include any of the slot characteristics described throughout the present disclosure, namely kerf, segment length, uncut angle, cut angle, upper slot angle, and lower slot angle.

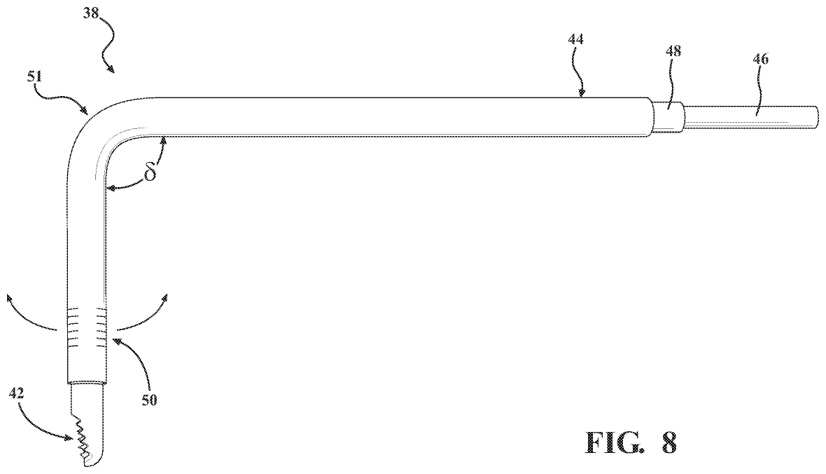

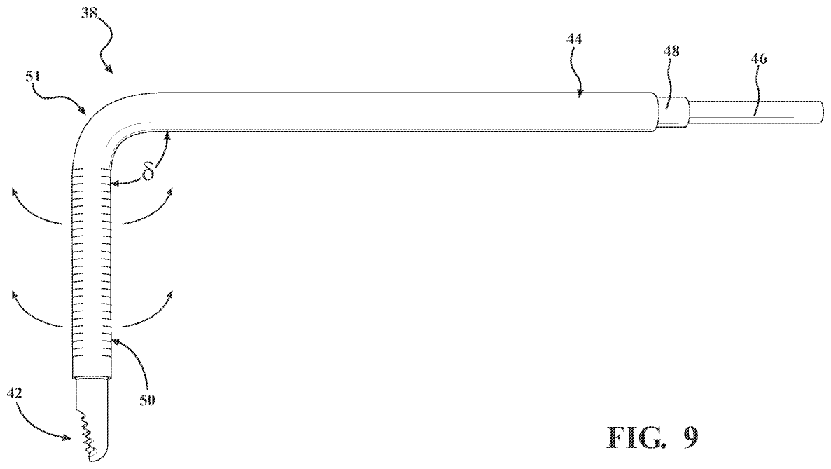

FIGS. 8 and 9 illustrate implementations of the tube assembly 38 in which there is an initial fixed prebend of a solid section 51 (i.e., not malleable). The initial fixed prebend may be at a predetermined angle (δ) within the range of approximately 60 to 120 degrees, more particularly within the range of approximately 80 to 100 degrees, and even more particularly approximately 90 degrees. The slotted region(s) 50 are positioned distal to the initial fixed prebend and may be of any suitable length based on the target anatomy. The slotted region 50 of FIG. 8 is shorter and may provide for a singular bend, whereas FIG. 9 is longer and may provide for a compound bend in addition to the initial fixed prebend.

Implementations with the initial fixed prebend may be particularly well suited for accessing the sinus, and more particularly the maxillary or frontal sinuses. For example, in instances in which the sinus may be partially or completely occluded, the surgeon may bend the slotted region 50 to reach more medial or lateral aspects of the inferior portion of the maxillary sinus. Furthermore, the initial fixed prebend may advantageously provide rigidity to the tube assembly 38 to account for any leveraging action that may occur between the cutting accessory 24 and the rigid anatomy (e.g., skull based). Still further, the initial fixed prebend may be considered more intuitive as to the direction of the bend as opposed to other implementations in which most of the length of the tube assembly 38 is malleable.

Referring again to FIG. 1 and FIG. 2, the tube assembly 38 may be a two-tube or three-tube arrangement. In the two-tube arrangement, the outer tube 44 includes the slotted region(s) 50 and further defines the cutting window 40. The inner tube 46 is driven by the motor to rotate within the outer tube 44. In the three-tube arrangement, the intermediate tube 48 is coaxially disposed between the outer tube 44 and the inner tube 46, and defines the cutting window 40. In certain implementations, the intermediate tube 48 facilitates rotational adjustment of the cutting window 40. The cutting accessory 24 may include an actuator 60 coupled to the outer hub 36, and operably coupled to the intermediate tube 48. The coupling may be facilitated by suitable gearing within the outer hub 36, for example, bevel gearing, worm gearing, or the like. FIG. 1 shows the actuator 60 as a barrel-styled thumbwheel configured to receive an input from the user grasping the surgical cutting instrument 20. Other suitable actuators may include a rotatable dial, pivotable lever, and the like. The input to the actuator 60 is configured to rotate the intermediate tube 48 relative to the outer tube 44, and allow the inner tube 46 to rotate the cutting window 40 about the longitudinal axis.

In the two-tube arrangement in which suction and irrigation is provided, it may be indicated to prevent egress of the irrigation fluid from the slotted region 50 of the outer tube 44. The cutting accessory 24 may include a first liner (not shown) coupled to the outer tube 44 and disposed over the slotted region 50. The first liner may be a heat-shrink tubing disposed over an outer surface of the outer tube 44, or a tubular jacket coupled to the outer surface of the inner surface of the outer tube 44. Likewise, it may be indicated to prevent ingress of the irrigation fluid from the irrigation path to the suction path defined by the inner tube 46. A second liner (not shown) may be coupled to and disposed over or within the flexible region(s) of the inner tube 46. In the three-tube arrangement, a third liner (not shown) may be provided and coupled to and disposed over or within the flexible region(s) of the intermediate tube 48.

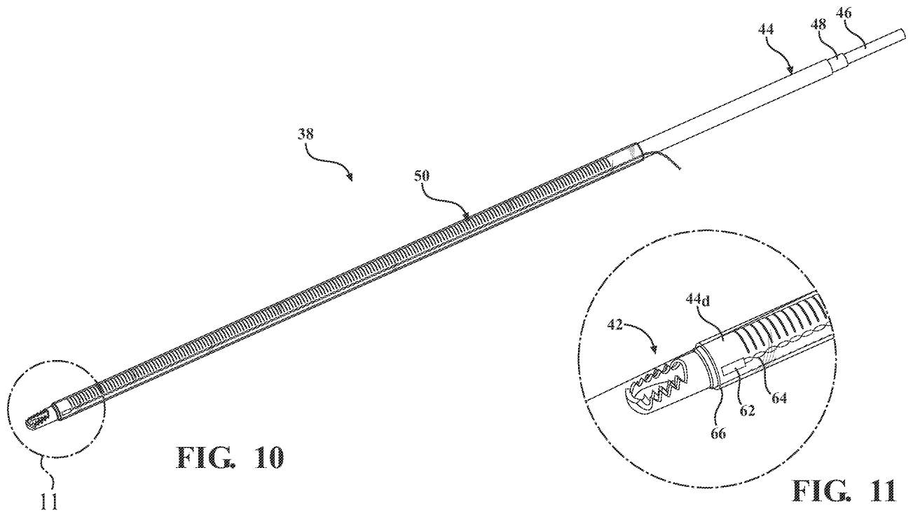

The navigation of surgical instruments is becoming increasingly commonplace in the modern surgical suite. Known devices enabling navigation are typically rigid such that a calibrated location of the shaft tip remains static relative to, for example, a tracking array coupled to the handpiece. Alternatively, certain devices enabling navigation may require calibrating and registering a tip to the tracking array. Such solutions fail to address the on-the-fly adjustment afforded by the tube assembly 38 of the present disclosure. In other words, requiring the user re-register the cutting tip 42 in navigation software after each time the tube assembly 38 is bent and rebent would be cumbersome. Therefore, in certain implementations, the cutting accessory 24 of the present disclosure overcomes such shortcomings by providing a sensor 62 (see FIG. 11) coupled to the tube assembly 38 and disposed distal to the malleable region. Referring to FIGS. 10 and 11, the sensor 62 is coupled to the rigid distal segment 44d; i.e., the tube head that is distal to the slotted region 50. The sensor 62 is positioned in a fixed spatial relationship relative to a predetermined point on the cutting tip 42, for example, a distalmost point. The sensor 62 may be an electromagnetic (EM) sensor, or other suitable tracking technology not requiring line-of-sight. As a result, regardless of the nature and quantity of the bends being imparted to the tube assembly 38, data indicative of the location of the cutting tip 42 that is transmitted from the sensor 62 to the navigation software remains sufficiently accurate, and further does not require re-registration after successive bending events.

A lead 64 is configured to couple the sensor 62 to electronic subcomponents (not shown) within the outer hub 36. The lead 64 may extend proximally from the sensor 62 and/or along the malleable spine 54 so as to limit strain with the bending of the tube assembly 38. The lead 64 may be a twisted wire pair to reduce interference from the sensor 62. A second sensor may be coupled on the opposing side of the outer tube 44 than that shown in FIG. 9 with a second lead extending along the opposing malleable spine.

The sensor 62 and the lead 64 may be secured to the outer tube 44 with a sheath 66. The sheath 66 may be the first liner previously mentioned, or alternatively, the sheath 66 may be in addition to the first liner. The sheath 66 may be a heat-shrink material, a polymeric jacket secured the outer tube 44, or the like, so as to fix the position of the sensor 62 and the lead 64 in a low-profile manner that does not obstruct visualization of the cutting tip 42 when viewed along the tube assembly 38.

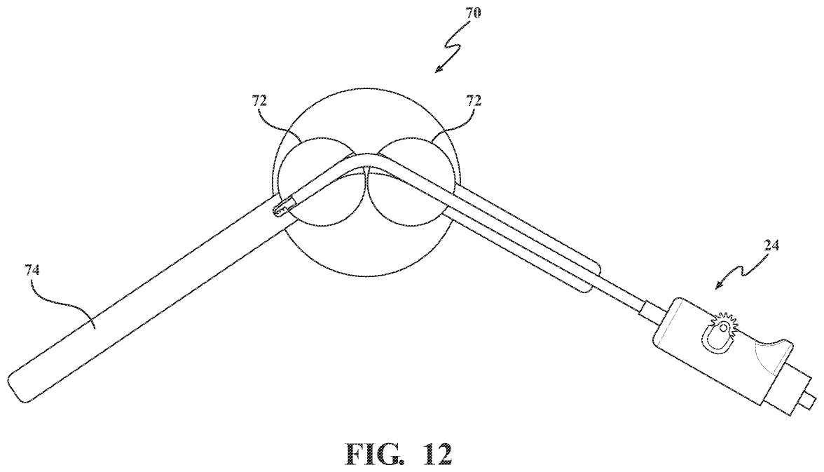

The bending of the tube assembly 38 may be done manually by the user. Additionally, or alternatively, a bending apparatus 70 as shown in FIG. 12 may be provided to assist the user. The bending apparatus 70 may be packaged in a kit with the cutting accessory 24. FIG. 12 shows an example of the bending apparatus 70 with the cutting accessory 24 disposed therein. The bending apparatus 70 may include opposing jigs 72. The jigs 72 define slots configured to be aligned with one another to permit the tube assembly 38 of the cutting accessory 24 to be inserted in the straight configuration. The user may insert the tube assembly 38 in a manner to position a desired location of the bend at an interface between the two jigs 72. The bending apparatus 70 may include at least one actuator 74 configured to receive an input from the user to rotate one or both of the jigs 72 relative to one another. The jigs 72 may be rotated in counterposing directions to impart the bend to the tube assembly 38, for example, as shown in FIG. 12. Indicia may be disposed on the jigs 72 or a backplate of the bending apparatus 70 to provide an indication of the bend angle being imparted to the tube assembly 38.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described. For example, the malleability need not be provided by the slots, but instead may be based on the materials forming the outer tube 44. In such an example, the tube assembly 38 may include malleable region(s) as opposed to slotted region(s) 50.

1. A cutting accessory configured to be removably coupled to a handpiece of a surgical cutting instrument including a motor, the cutting accessory comprising:
an outer hub configured to be coupled with the handpiece;
a drive hub rotatably disposed within the outer hub and configured to be operably coupled to the motor; and
a tube assembly comprising an outer tube coupled to and extending distally from the outer hub, an intermediate tube coaxially disposed within the outer tube and extending distally from the outer hub, an inner tube coupled to the drive hub and coaxially disposed within the intermediate tube, and a cutting tip disposed on the inner tube, wherein each of the intermediate tube and the inner tube comprises at least one flexible region,
wherein the outer tube is formed with at least one slotted region in which a series of slots form a malleable spine configured to be bent and/or rebent by a user to a shaped configuration and maintain the tube assembly in the shaped configuration.

2. The cutting accessory of claim 1, wherein the at least one slotted region further comprises a proximal slotted region, and wherein the outer tube is further formed with a distal slotted region that is spaced apart from the proximal slotted region to define a rigid proximal segment, a rigid intermediate segment, and a rigid distal segment of the tube assembly.

3. The cutting accessory of claim 2, wherein the series of slots of the proximal slotted region are formed to orient the malleable spine of the proximal slotted region on a first rotational orientation, and wherein the series of slots of the distal slotted region are formed to orient the malleable spine of the distal slotted region in a second rotational orientation different than the first rotational orientation.

4. (canceled)

5. The cutting accessory of claim 2, wherein spacing between adjacent slots of the series of slots of the proximal slotted region is greater than spacing between adjacent slots of the series of slots of the distal slotted region.

6. The cutting accessory of claim 1, wherein the tube assembly is further shaped with a rigid proximal prebend, and wherein the slotted region further comprises a distal slotted region spaced apart from the rigid proximal prebend.

7. (canceled)

8. The cutting accessory of claim 1, wherein the malleable spine is shaped with an initial prebend at a predetermined angle and configured to be bent and/or rebent to the shaped configuration about the predetermined angle.

9. The cutting accessory of claim 1, wherein the slotted region extends along nearly an entirety of an exposed length of the outer tube.

10. The cutting accessory of claim 1, further comprising an actuator coupled to the outer hub and the intermediate tube, wherein the actuator is configured to receive an input from the user to rotate a cutting window defined by the intermediate tube relative to the outer tube.

11. The cutting accessory of claim 1, wherein the at least one flexible region of the intermediate tube is formed by cut geometries that are different than the series of slots of the slotted region of the outer tube.

12. The cutting accessory of claim 1, further comprising:
a first liner coupled to the outer tube and disposed over the slotted region;
a second liner coupled to and disposed over the at least one flexible region of the inner tube; and
a third liner coupled to and disposed over the at least one flexible region of the intermediate tube.

13. (canceled)

14. (canceled)

15. A cutting accessory configured to be removably coupled to a handpiece of a surgical cutting instrument including a motor, the cutting accessory comprising:
an outer hub configured to be coupled with the handpiece;
a drive hub rotatably disposed within the outer hub and configured to be operably coupled to the motor; and
a tube assembly comprising an outer tube coupled to and extending distally from the outer hub, an inner tube coupled to the drive hub and coaxially disposed within the outer tube, and a cutting tip disposed on the inner tube, wherein the inner tube comprises at least one flexible region,
wherein the outer tube is formed with a proximal malleable region and a distal malleable region that is spaced apart from the proximal malleable region to define a rigid proximal segment, a rigid intermediate segment, and a rigid distal segment of the tube assembly, and wherein the proximal and distal malleable regions are configured to be independently bent and/or rebent by a user to a shaped configuration, and maintain the tube assembly in the shaped configuration.

16. The cutting accessory of claim 15, wherein each of the proximal and distal malleable regions are formed by a series of slots extending circumferentially about a portion of the outer tube, wherein the series of slots of the proximal malleable region form a proximal malleable spine oriented in a first rotational orientation, and wherein the series of slots of the distal malleable region form a distal malleable spine oriented in a second rotational orientation different than the first rotational orientation.

17. (canceled)

18. (canceled)

19. The cutting accessory of claim 16, wherein the tube assembly is formed in an initial straight configuration in which the proximal and distal malleable spines extend along a longitudinal axis, wherein the proximal and distal malleable spines are configured to be bent and/or rebent to the shaped configuration about the longitudinal axis.

20. The cutting accessory of claim 16, wherein at least one of the proximal and distal malleable spines are shaped with an initial prebend at a predetermined angle and configured to be bent and/or rebent to the shaped configuration about the predetermined angle.

21. The cutting accessory of claim 15, wherein spacing between adjacent slots of the series of slots of the proximal slotted region is greater than spacing between adjacent slots of the series of slots of the distal slotted region.

22. The cutting accessory of claim 15, wherein the tube assembly further comprises an intermediate tube coaxially disposed between the outer tube and the inner tube.

23. A cutting accessory configured to be removably coupled to a handpiece of a surgical cutting instrument including a motor, the cutting accessory comprising:
an outer hub configured to be coupled with the handpiece;
a drive hub rotatably disposed within the outer hub and configured to be operably coupled to the motor; and
a tube assembly comprising an outer tube coupled to and extending distally from the outer hub, an inner tube coupled to the drive hub and coaxially disposed within the outer tube, and a cutting tip disposed on the inner tube, wherein the inner tube comprises at least one flexible region,
wherein the outer tube is formed with a proximal slotted region and a distal slotted region in which a series of slots respectively form a proximal malleable spine and a distal malleable spine configured to be independently bent and/or rebent by a user to a shaped configuration, and maintain the tube assembly in the shaped configuration, and wherein spacing between adjacent slots of a series of slots of the proximal slotted region is greater than spacing between adjacent slots of a series of slots of the distal slotted region.

24. The cutting accessory of claim 23, wherein the tube assembly further comprises an intermediate tube coaxially disposed between the outer tube and the inner tube.

25-28. (canceled)

29. The cutting accessory of claim 1, further comprising:
a sensor positioned distal to the slotted region or the malleable region;
a lead coupled to and extending proximally from the sensor, wherein the lead is configured to be coupled with electronic subcomponents of the outer hub; and
a sheath coupling the sensor and the lead to the outer tube.

30. The cutting accessory of claim 29, wherein the lead extends along the malleable spine of the slotted region.
